SEQUENCE LISTING (2 sequences)

SEQ ID NO: 1 - DNA, 2217 bp, Artificial Sequence
  Staphylococcus aureus cadA gene with BamH I and Xba I restriction sites and plant specific translation signals
tctagatttaccaccatgtctgaacaaaaggttaaactaatggaagaagaaatgaacgtctatcgggtccaaggatttacatgtgcaaattgtgcaggaaagtttgagaaaaatgttaaaaagattccaggcgttcaggacgcaaaagtaaactttggcgcttctaaaattgatgtatatggaaatgcatcggttgaagaacttgaaaaagcaggtgcttttgagaatctaaaagtatctcctgaaaaactagcgaatcaaacgatacaaagggttaaagatgacactaaggctcataaagaagagaaaacaccattttataaaaaacatagtacattgctgtttgccacactactaattgcttttggttacctttctcactttgtaaatggagaagataacctcgtaacttccatgttatttgtaggttctattgtaattggcggatattcattatttaaagtcggttttcaaaatttgatacgctttgatttcgacatgaaaaccctgatgaccgttgccgttattggagctaccattattggtaaatgggcagaggcatctattgttgttattctctttgcaatcagtgaagcacttgaacgcttctctatggacagatcaagacaatccatacgttcattgatggatatcgccccaaaagaagcactagttagacgaaatggtcaggaaataataatccatgtggacgatatcgctgtgggtgatatcatgattgtcaaaccaggggagaaaattgccatggatggaatcattgtgaatggcttgtcggctgtcaatcaggcagctataacaggagaatctgttcccgtctccaaagcggtagatgacgaagtatttgcaggtacgcttaacgaagagggactaattgaagtaaaaatcaccaaatacgtagaagatacaaccattaccaagattattcatcttgttgaagaagcacaaggggagcgtgctccagcccaagcattcgttgataaatttgcgaaatactacactccgatcattatggttattgcagccttggttgcagtcgttccacccctattctttggtggcagttgggatacatgggtttatcaaggattagcagttcttgtagttggatgtccttgtgcattagttatttctactccaatctcgattgtctcggcaattggaaatgcagcgaaaaaaggtgtgttggttaaaggtggtgtctatctcgagaaattaggagccattaagacagtcgcatttgataaaacaggaacactgacaaaaggtgtaccagtggtaacagattttgaagtattaaatgaccaagtggaagaaaaagagctattctctatcattacagctttagaatatcgttcacaacatccacttgcttcagcaataatgaaaaaggcagagcaagataatatcccttattctaatgtacaagtggaagaattcacttcgattactgggcgaggtataaaagggattgtaaacggaactacttactatattggaagcccaaaacttttcaaggaattaaatgtttccgattttagccttgggtttgaaaacaatgtgaaaatcctacaaaaccaaggaaaaacagccatgattattggaacggaaaaaacaattctcggcgtaattgccgttgcagatgaggttcgtgaaacaagtaaaaatgtgattcaaaaacttcatcagttaggtatcaagcaaacaattatgctgacaggtgataatcaaggtactgcaaatgcaatcggtacacatgtaggcgtttctgatattcagtctgaattgatgccacaggataaattagattatattaaaaaaatgcaatcggagtatgataatgtagctatgattggcgatggcgttaatgatgctccagcacttgctgcatctactgttggaattgcaatgggcggtgctggaacggatactgcaattgaaacagctgatattgcattaatgggagatgatttaagtaagcttccatttgcagtaagactcagtcgaaaaactttaaatatcattaaagctaacatcacttttgctatcggaattaaaataattgccttactattagttatcccgggatggttaaccctttggatagcgattctttccgatatgggagctactattttggtagcattaaatagtttacgactgatgagagtgaaggataaataggtaatgatgtttggatcc

SEQ ID NO: 2 - protein, 727 aa, Artificial Sequence
  Staphylococcus aureus cadA gene with BamH I and Xba I restriction sites and plant specific translation signals
MSEQKVKLMEEEMNVYRVQGFTCANCAGKFEKNVKKIPGVQDAKVNFGASKIDVYGNASVEELEKAGAFENLKVSPEKLANQTIQRVKDDTKAHKEEKTPFYKKHSTLLFATLLIAFGYLSHFVNGEDNLVTSMLFVGSIVIGGYSLFKVGFQNLIRFDFDMKTLMTVAVIGATIIGKWAEASIVVILFAISEALERFSMDRSRQSIRSLMDIAPKEALVRRNGQEIIIHVDDIAVGDIMIVKPGEKIAMDGIIVNGLSAVNQAAITGESVPVSKAVDDEVFAGTLNEEGLIEVKITKYVEDTTITKIIHLVEEAQGERAPAQAFVDKFAKYYTPIIMVIAALVAVVPPLFFGGSWDTWVYQGLAVLVVGCPCALVISTPISIVSAIGNAAKKGVLVKGGVYLEKLGAIKTVAFDKTGTLTKGVPVVTDFEVLNDQVEEKELFSIITALEYRSQHPLASAIMKKAEQDNIPYSNVQVEEFTSITGRGIKGIVNGTTYYIGSPKLFKELNVSDFSLGFENNVKILQNQGKTAMIIGTEKTILGVIAVADEVRETSKNVIQKLHQLGIKQTIMLTGDNQGTANAIGTHVGVSDIQSELMPQDKLDYIKKMQSEYDNVAMIGDGVNDAPALAASTVGIAMGGAGTDTAIETADIALMGDDLSKLPFAVRLSRKTLNIIKANITFAIGIKIIALLLVIPGWLTLWIAILSDMGATILVALNSLRLMRVKDK

(19) United States
(12) Patent Application Publication
(10) Pub. No.: US 2004/0055040 A1
(43) Pub. Date: Mar. 18, 2004

(54) Genetically modified plants and plant cells comprising heterologous heavy metal transport and complexation proteins

(76) Inventors: Brigitte Borremans, Bierbeek (BE); Nathalie Bousmans, Sint-Gensius-Rode (BE); Michel Jacobs, Brussels (BE); Maximilien Mergeay, Retie (BE); Daniel Van Derj Lelie, Bluuo Geel (BE); Jaco Vangronsveld, Deipenbeek (BE); Sirpa Orvokki Karenlampi, Kuopio (FI); Arja Irmeli Tervahauta, Leppavirta (FI)

Correspondence Address: MERCHANT & GOULD PC, P.O. BOX 2903, MINNEAPOLIS, MN, 55402-0903, US

(21) Appl. No.: 10/239,339

(22) PCT Filed: Mar. 22, 2001
(86) PCT No.: PCT/BE01/00049

(30) Foreign Application Priority Data
Mar. 22, 2000 (EP) 00870051.0

(51) Int. Cl. A01H 1/00; C12N 15/82

(52) U.S. Cl. 800/289

(57) ABSTRACT

The present invention relates to genetically modified plants and plant cells, comprising nucleotide sequences encoding heterologous heavy metal transport protein.

### FIELD OF THE INVENTION

The present invention is in the field of genetically modified plants and plants cells having improved heavy metal tolerance and accumulation due to increased plant growth and biomass production based upon the expression of exo-cytoplasmic heavy metal resistance system (efflux and complexation).

More particularly, the present invention is related to genetically modified plants and plant cells, comprising nucleotide sequences encoding heterologous heavy metal transport proteins and exocytoplasmic metal binding proteins of various origins.

### BACKGROUND OF THE INVENTION AND STATE OF THE ART

Heterologous nucleic acid sequences, coding for heavy metal resistance, were functionally expressed in plants, to improve their tolerance against these toxic elements. The heterologous heavy metal resistance genes, in casu represent either heavy metal efflux systems or functions involved in heavy metal sequestration.

Until present, only cytoplasmic functions that provide increased heavy metal resistance were expressed in plants.

1. Expression of Heterologous Metallothionein and Phytochelatines in Plants

Metallothioneins and phytochelatines, which are rich in cystein sulfhydryl residues that bind and sequester heavy metal ions in very stable complexes (Karin, 1985), are found in eukaryotic organisms, but recently also in Synechococcus. Various MT genes—mouse MTI, human MTIA (alpha domain), human MTII, Chinese hamster MTII, yeast CUP1, pea PsMTA—have been transferred to tobacco, cauliflower or Arabidopsis thaliana (Lefebre et al., 1987; Maiti et al., 1988, 1989, 1991; Misra and Gedamu, 1989; Evans et al., 1992; Yeargan et al., 1992; Brandle et al., 1993; Pan et al., 1993; Elmayan and Tepfer, 1994; Hattori et al., 1994; Pan et al., 1994a, b; Hasegawa et al., 1997). As a result, varying degrees of enhanced Cd tolerance have been achieved, being maximally 20-fold compared with the control. Metal uptake levels were not dramatically changed; in some cases there were no differences, in others maximally 70% less or 60% more Cd was taken up by the shoots or leaves. Only one study has been reported on a transgenic plant generated with MT of plant origin. When pea (Pisum sativum) MT-like gene PsMTA was expressed in Arabidopsis thaliana, more Cu (several-fold in some plants) accumulated in transformed than in control plants (Evans et al., 1992).

2. Heterologous Expression of Heavy Metal Reduction

The only example known is the mer operon of Tn21 of Shigella flexneri, whose expression in plants results in the reduction mercury (Hg²⁺) in its metallic form (Hg⁰). This metallic mercury is volatilized out of the cell (Rugh et al. 1996).

### AIMS OF THE INVENTION

The present invention aims to provide a new way in obtaining plants and plant cells with improved heavy metal tolerance characteristics, and possibly heavy metal accumulation.

Another aim of the present invention is to provide such plants and plant cells which allow increased heavy metal resistance for revegetation and phytostabilisation of heavy metal contaminated sites.

A further aim of the present invention is to provide plants and plant cells, characterised by increased heavy metal accumulation combined with increased heavy metal tolerance which allow phytoextraction of heavy metals (inclusive rhizofiltration).

A last aim of the present invention is to provide a method which results in the possibility to improve important agriculture crop species with high biomass production in their heavy metal tolerance and accumulation.

### SUMMARY OF THE INVENTION

The present invention is related to genetically modified plant and plant cell having improved (induced or increased) heavy metal resistance, comprising at least one nucleotide sequence encoding one or more heterologous heavy metal transport and/or sequestration proteins of various prokaryotic or eukaryotic origins.

Said transporters are preferably membrane proteins, which result in reduced toxicity due to the efflux of heavy metals from the cells and being preferably selected from the group consisting of P-type ATPases, 3 component efflux pumps, ABC transporters and CDF proteins (Cation Diffusion Facilitator proteins).

The family of the P-type ATPases is preferred, because of their advantage that for functional resistance only one protein is required.

Said proteins are found in both prokaryotic and eukaryotic organisms including plants.

Another advantage of said transporters is found as resistance mechanisms against many toxic trace elements of environmental concern, such as copper, cadmium, lead, zinc and silver.

Unexpectedly, it was not necessary to make structural changes in the coding sequence of said proteins, like it is necessary for the merA gene in order to obtain functional expression in plants (Rugh et al., 1996).

Preferably, the gene incorporated in the plants or plant cells is a gene encoding a bacterial P-type ATPase, preferably the cadmium ATPase, such as the cadA gene.

Another aspect of the present invention is related to a method for inducing (or improving) increased heavy metal resistance into a plant or a plant cell, said method comprising the following steps:

preparing at least one nucleotide sequence encoding one or more heterologous heavy metal transport and/or sequestration proteins, operably linked to one or more regulatory sequences active into a plant,

transforming a plant or plant cell with said nucleotide sequence and,

possibly regenerating a (transgenic) plant from the transformed plant or plant cell.

According to a second embodiment of the present invention, the system is based upon a prokaryotic heavy metal sequestration system, such as the pcoA family protein (more preferably the pcoA gene).

The various nucleotide sequences encoding heterologous heavy metal transport proteins can be deleted partially from non-specific nucleotide sequences which are not involved in efficient heavy metal transport or accumulation.

Said genetic sequences could be incorporated in a vector for the transfection of said plants or plant cells, such as the pBI121 vector, as described in the FIG. 1, said vector being advantageously an E. coli/Agrobacterium/plant shuttle vector, said vector comprising preferably a CaMV 35S promoter (a strong promoter constitutively expressed in plants).

Preferably, the system was introduced in the plants, such system allowing the transformation of plants with the Agrobacterium tumefaciens technology.

The present invention can be used for phytoremediation of contaminated sites, or for the preparation of a medicament or food/feed supplements containing trace elements.

The plant or plant cell according to the invention has various applications, especially in the field of phytoremediation of contaminated sites with heavy metal (such as areas having grounds contaminated with heavy metals or aquatic or semi-aquatic areas contaminated with heavy metals). The phytoremediation of contaminated sites comprises usually the step of revegetation, phytostabilisation, phytoextraction of soil and/or water contaminated with trace elements of said heavy metal.

Another application of the plant or plant cell according to the invention is a medical and agro-industrial application, wherein said plant or plant cell are natural sources for trace elements.

Therefore, a last aspect of the present invention is related to a pharmaceutical composition of food or feed compositions or additives, containing said plant or plant cells with heavy metal trace elements.

### SHORT DESCRIPTION OF THE DRAWINGS

FIG. 1 is a schematic representation of the cloning of cadA in pBI121.

FIG. 2 is a leaf disk-test with Nt WT SR1 (wild type), Nt PBI14 (pBI121) and Nt Cd 309 (pBI121-cadA) on 350 μM Cd and control medium without Cd.

FIG. 3 represents the regeneration and growth of Nt WT SR1 (wild type), Nt PBI14 (pBI121) and Nt Cu122 (pBI121-pcoA) on 100 μM Cu, the plant growth being shown from above (left) and top (right).

### DETAILED DESCRIPTION OF THE INVENTION

Heterologous Expression of cadA

The heavy metal efflux system was cadA, a member of the P-type heavy metal efflux ATPase family of proteins found both in prokaryotic and eukaryotic organisms. P-type ATPases are all cation pumps, either for uptake, for efflux or for cation exchange. These enzymes have a conserved aspartate residue that is transiently phosphorylated from ATP during the transport cycle, hence the name ‘P-type’ ATPase (Silver et al., 1993).

The cadA gene from Staphylococcus aureus was amplified by PCR and cloned in the pBI121 vector.

During PCR, appropriate plant specific translation signals were added as well as XbaI and BamHI restriction sites, allowing cloning of the insert in the correct orientation.

The cadA fragment was cloned in the Escherichia coli/Agrobacterium/plant shuttle vector pBI121. In this vector, cadA expression is derived from the CaMV35S promotor, a strong promoter constitutively expressed in plants. The system was introduced in the plant Nicotiana tabacum cv. Petit Havana line SR1 via an Agrobacterium tumefaciens transformation (Horsch et al., 1985). The selection marker used was kanamycine.

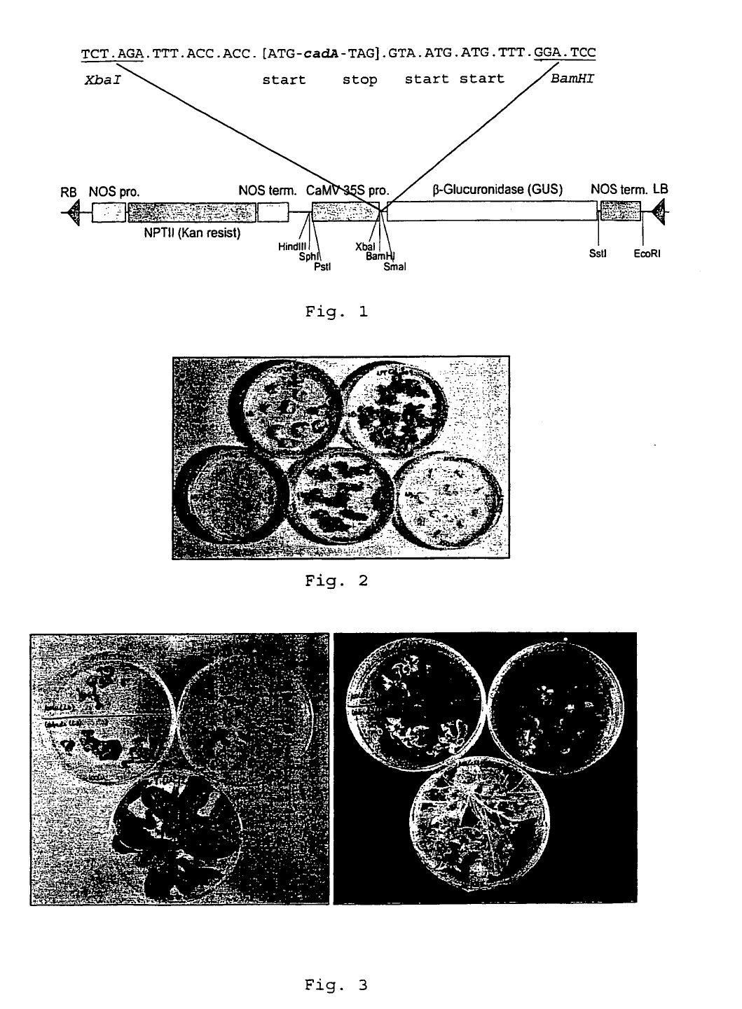

Kanamycine resistant transformants were obtained after transformation. All the kanamycine resistant transformants tested showed an increased resistance to cadmium (tested by a leaf disk assay) compared to the wild type and transformant with the pBI121 vector without gene (FIG. 1). This proves that the CadA P-type ATPase can be functionally expressed in plants, resulting in an increased resistance of the plant to the trace element (in casu cadmium).

It can be expected that for other members of the P-type ATPase family, which form a family of closely related proteins (both structural and functional) the same positive effect on resistance to specific trace elements will be found. Until present, P-type ATPases from both prokaryotic and eukaryotic have been identified that were found to interact with Zn, Cd, Pb, Cu and Ag (see table 1). It can not be excluded that P-type ATPases, encoding resistance to other trace elements including radioisotopes, will be identified.

TABLE 1
different representatives of the family of P-type
ATPases, from prokaryotic and eukariotic origin, which
encode resistance against trace elements
such as Zn, Cd, Pb, Cu and Ag.

| | Sequence | | |
|---|---|---|---|
| Gene | ID | Metals | Reference |
| CadA | P20021 | Cd, Zn and | Nucifora et al. 1989 |
| | | Pb | Rensing et al. 1998 |
| ZntA | P37617 | Zn and Pb | Rensing et al. 1997 |
| | | | Rensing et al. 1998 |
| CopF | Non | Cu | van der Lelie and |
| | available | | Borremans |
| | | | unpublished |
| PbrA | Not | Pb | Borremans et al, 2000 |
| | available | | |
| SilP | AF067954, | Ag | Gupta et al, 1999 |
| | nucleotide | | |
| | sequence | | |
| | sil operon | | |
| Menkes' | Q04656 | Cu | Vulpe et al. 1993 |
| disease | | | |
| Wilsons' | 1J08344 | Cu | Pethrukin et al. 1993 |
| disease | | | |

Heterologous Expression of pcoA

The other heavy metal resistance system is involved in exo-cytoplasmic heavy metal sequestration. The tested gene here was pcoA from Escherichia coli (Brown et al., 1995), which was also cloned in pBI121 and introduced in Nicotiana tabacum through an Agrobacterium tumefaciens transformation in a way similar as described for cadA. Kanamycine resistant transformants were obtained after transformation. All the kanamycine resistant transformants tested showed an increased resistance to copper (tested by a leaf disk assay) compared to the wild type and transformant with the pBI121 vector without gene (FIG. 3).

The pcoA protein has many closely related members, found to be involved in resistance against Cu. In addition, other proteins of these copper resistance determinants have also been shown to be involved in Cu sequestration, such as PcoC/CopC and CopE. These proteins, although different in structure, are also active in the bacterial periplasm and possess similar heavy metal binding sites as pcoA. In addition, a CopE like protein, referred to as SilE, was identified in the Salmonella sil operon encoding for Ag-resistance. The potential genes whose heterologous expression can result in improved resistance, are summarised in table 2.

| Genes | Sequence ID | Metals | References |
|---|---|---|---|
| cop operon (copA, C) | M19930 | Cu | Mellano and |
| e.g. of Pseudomonas | | | Cooksey |
| syringae | | | (1988) |
| pco operon (pcoA, C) | G619126 | Cu | Brown et al., |
| of e.g. E. coli | | | 1995 |
| PcoE | X83541 | Cu | Brown et al., |
| | | | 1995 |
| sil operon of | AF067954, | Ag | Gupta et al., |
| Salmonella | nucleotide | | 1999 |
| | sequence | | |
| | sil operon | | |

Use of the Genetically Modified Plants or Plant Cells According to the Invention:

Plants or plant cells according to the present invention can be used in several applications. It is clear that a plant according to the invention can be used for phytoremediation of contaminated sites, for revegetation, phytostabilisation, phytoextraction of contaminated soils and/or water. While the genetic modification allow the plants to grow in such contaminated environments, they will accumulate trace elements and remove them from the soil and/or water.

Another possible application is to grow said genetically modified plants or plant cells on a medium (solid or fluid) containing certain trace elements necessary or beneficial for human and animal health.

It would then be possible to prepare food/feed supplements from these plants containing beneficial trace elements which can be readily adsorbed. Further, the plants could serve as a basis for the preparation of a medicament.

### REFERENCES

Silver S. et al. (1993). Molecular Microbiology 10(1): 7-12.

Horsch R. B. et al. (1985). Science 227: 1229-1231.

Karin M (1985). Metallothioneins: Proteins in search of function; Cell 41, 9-10.

Brown N. L. et al. (1995). Molecular Microbiology 17(6): 1153-1166.

Mellano, M. A. et al. (1988). J. Bacteriol. 170: 2879-2883.

Vulpe C. D., et al. (1993). Nature genet. 3: 7-13.

Petrukhin, K. et al. (1993). Nature Genet. 5 (4): 338-343.

Rugh C. L. et al. (1996). Proc. Natl. Acad. Sci. USA 93: 3182-3187.

Lefebvre, D. D. et al, 1987. Bio/Technology 5, 1053-1056.

Maiti, I. B. et al, 1988. Biochemical and Biophysical Research Communications 150, 640-647.

Maiti, I. B. et al, 1989. Plant Physiology 91, 1020-1024.

Maiti, I. B. et al. 1991. Plant Science 76, 99-107.

Misra, S. et al. 1989. Theoretical and Applied Genetics 78, 161-168.

Evans, K. M. et al 1992. Implications for PsMTA function. Plant Molecular Biology 20, 1019-1028.

Yeargan, R. et al 1992. Transgenic Research 1, 261-267.

Nucifora G. et al. (1989) Proc. Natl. Acad. Sci. U.S.A. 86 (10): 3544-3548.

Rensing C., et al. (1998). J. Biol. Chem. 273: 32614-32617.

Rensing C., et al. (1997). Proc. Natl. Acad. Sci. U.S.A. 94 (26): 14326-14331.

Gupta, A., et al. 1999. Nature Medicine 5: 183-188.

1. Genetically modified plant or plant cell having an increased heavy metal resistance and comprising at least one nucleotide sequence encoding a protein selected from the group consisting of heterologous heavy metal transporter or sequestration proteins.

2. The genetically modified plant or plant cell as in claim 1, characterised in that said nucleotide sequence is a prokaryotic nucleotide sequence.

3. The genetically modified plant or plant cell as in claim 1 or 2, wherein the heavy metal transporter is a transporter selected from the group consisting of P-type ATPases, 3 components efflux pumps, ABC transporters or Cation Diffusion Facilitor proteins.

4. The genetically modified plant or plant cell according to the claim 3, wherein the transporter is a cadmium ATPase.

5. The genetically modified plant or plant cell according to the claim 3 or 4, wherein the nucleotide sequence is cadA or a portion thereof allowing heavy metal transport.

6. The genetically modified plant or plant cell according to the claim 1, wherein the heavy metal sequestration protein belongs to the copA family.

7. Method for inducing or increasing heavy metal resistance into a plant or plant cell, comprising the steps of:
preparing a nucleotide sequence encoding a protein selected from the group consisting of heterologous heavy metal transporters or sequestration proteins, being operably linked to one or more regulatory sequences active into a plant,
transforming a plant or plant cell with said nucleotide sequence, and
possibly regenerating a plant from the transformed plant cell.

8. Use of the genetically modified plant or plant cell according to any of the preceding claims 1 to 6 for the phytoremediation of contaminated heavy metals.

9. The use according to the claim 8, wherein the phytoremediation of contaminated sites is selected from the group consisting of revegetation, phytostabilisation, phytoextraction of soils and/or water contaminated with trace elements.

10. Pharmaceutical composition comprising an adequate pharmaceutical carrier and a sufficient amount of the genetically modified plant or plant cell according to any of the preceding claims 1 to 6.

11. Feed or food composition or additive comprising the genetically modified plant or plant cell according to any of the preceding claims.
